(19) United States
(12) Patent Application Publication
(10) Pub. No.: US 2025/0266251 A1
(43) Pub. Date: Aug. 21, 2025

(54) APPARATUS FOR FABRICATING DISPLAY PANEL

(71) Applicant: Samsung Display Co., LTD., Yongin-si (KR)

(72) Inventors: Eun Ju SONG, Yongin-si (KR); Hun Jung YI, Yongin-si (KR)

(73) Assignee: Samsung Display Co., LTD., Yongin-si (KR)

(21) Appl. No.: 19/049,499

(22) Filed: Feb. 10, 2025

(30) Foreign Application Priority Data
Feb. 16, 2024 (KR) 10-2024-0022537

(51) Int. Cl. H01J 37/32; G01N 21/31

(52) U.S. Cl. CPC H01J 37/32972; G01N 21/31; H01J 37/32954; H01J 37/32981; H01J 2237/3321; H01J 2237/3323

(57) ABSTRACT

The disclosure relates to an apparatus for fabricating a display panel. According to an embodiment of the disclosure, an apparatus for fabricating a display panel, comprising a thin film deposition device forming a thin film on a fabricating substrate, and a thin film characteristic detection device generating optical emission spectroscopy data by analyzing a spectrum of light emitted during a thin film deposition process of the thin film deposition device and detecting thin film characteristic information of the thin film formed on the fabricating substrate by analyzing the optical emission spectroscopy data in real time, wherein the thin film characteristic detection device detects the thin film characteristic information including a hydrogen concentration of the thin film formed on the fabricating substrate by analyzing a wavelength distribution of the light, an intensity distribution of the light, and a plurality of plasma characteristic information from the optical emission spectroscopy data.

### CROSS-REFERENCE TO RELATED APPLICATION(S)

This application claims priority to and benefits of Korean Patent Application No. 10-2024-0022537 under 35 U.S.C. 119, filed on Feb. 16, 2024, in the Korean Intellectual Property Office (KIPO), the entire contents of which are incorporated herein by reference.

### BACKGROUND

### 1. Technical Field

The disclosure relates to an apparatus for fabricating a display panel.

### 2. Description of the Related Art

As the information society develops, the demand for display devices for displaying images has increased and diversified. For example, display devices have been applied to various electronic devices such as smartphones, laptop computers, navigation devices, and smart televisions.

The display device may be a flat panel display device such as an organic light emitting display device using organic light emitting diodes, a quantum dot light emitting display device including quantum dot light emitting layers, an inorganic light emitting display device including inorganic semiconductors, and a micro or nano light emitting display device using micro or nano light emitting diodes.

When display panels of the display devices are fabricated, a process of depositing and patterning photoresists or organic or inorganic insulating materials on a transparent insulating substrate made of silicon, glass, or the like, is required. In particular, after a thin film is deposited on the insulating substrate, an inspection process for confirming whether or not the deposited thin film has been formed at a target thickness is performed. To this end, a process of measuring the thickness of the deposited thin film may be performed using a separate measurement facility.

Since a deposition process is performed several times in order to fabricate the display panel, the process of measuring the thickness of the thin film formed on the insulating substrate should be performed several times. Accordingly, a time required for a fabricating process of the display panel is delayed and efficiency of the deposition process is reduced.

### SUMMARY

Aspects of the disclosure provide an apparatus for fabricating a display panel capable of quickly and accurately detecting a deposition thickness and a refractive index of a thin film deposited on a transparent insulating substrate (hereinafter referred to as a fabricating substrate), a hydrogen concentration in the thin film, and the like, in real time.

Aspects of the disclosure also provide an apparatus for fabricating a display panel capable of confirming plasma characteristics and thin film deposition characteristics and correcting a thin film deposition process in real time by detecting and analyzing optical emission spectroscopy data in real time during the thin film deposition process.

However, aspects of the disclosure are not restricted to those set forth herein. The above and other aspects of the disclosure will become more apparent to one of ordinary skill in the art to which the disclosure pertains by referencing the detailed description of the disclosure given below.

According to an embodiment of the disclosure, an apparatus for fabricating a display panel may include a thin film deposition device forming a thin film on a fabricating substrate, and a thin film characteristic detection device generating optical emission spectroscopy data by analyzing a spectrum of light emitted during a thin film deposition process of the thin film deposition device and detecting thin film characteristic information of the thin film formed on the fabricating substrate by analyzing the optical emission spectroscopy data in real time. The thin film characteristic detection device may detect the thin film characteristic information including a hydrogen concentration of the thin film formed on the fabricating substrate by analyzing a wavelength distribution of the light, an intensity distribution of the light, and a plurality of plasma characteristic information from the optical emission spectroscopy data.

According to an embodiment of the disclosure, the thin film deposition device may include a chamber forming a space for performing a chemical vapor deposition process, a loading plate disposed in an internal space of the chamber and seating and fixing the fabricating substrate, a gas supplier supplying a deposition source gas and a carrier gas to the internal space of the chamber, and a plasma process controller applying high-frequency power to an inside of the chamber and controlling a plasma deposition process.

According to an embodiment of the disclosure, the thin film characteristic detection device may include a full-range spectroscopy module splitting and refracting the light emitted from an inside of a chamber of the thin film deposition device according to a spectrum wavelength band, a diffraction module reflecting the light refracted from the full-range spectroscopy module, an incident light filter module band-pass-filtering the light reflected from the diffraction module in a wavelength band, a photosensitivity detection module generating the optical emission spectroscopy data based on a wavelength distribution and an intensity distribution of the light for each wavelength band of the light filtered through the incident light filter module, and an optical data analysis device analyzing the wavelength distribution of the light, the intensity distribution of the light, and the plurality of plasma characteristic information from the optical emission spectroscopy data and detecting the thin film characteristic information including at least one of deposition thickness information, refractive index information, and hydrogen concentration information of the thin film formed on the fabricating substrate using calculation equations.

According to an embodiment of the disclosure, the optical data analysis device may include a plasma characteristic extraction module detecting the plurality of plasma characteristic information including the wavelength distribution of the light, the intensity distribution of the light, temperature characteristic change information of an inert gas, and molecule temperature characteristic change information of the inert gas from the optical emission spectroscopy data, a hydrogen concentration detection module calculating a hydrogen concentration of the thin film formed on the fabricating substrate in real time by using the optical emission spectroscopy data, the plurality of plasma characteristic information, and a hydrogen concentration detection calculation equation, and a deposition film characteristic detection module calculating a thickness of the thin film formed on the fabricating substrate in real time by using deposition environment information including temperature information and pressure information in the chamber, the plurality of plasma characteristic information, and a thin film thickness detection calculation equation.

According to an embodiment of the disclosure, the thin film characteristic detection device may include a photosensitivity detection module generating the optical emission spectroscopy data based on a wavelength distribution and an intensity distribution of the light for each wavelength band emitted from an inside of a chamber of the thin film deposition device, and an optical data analysis device analyzing the wavelength distribution of the light, the intensity distribution of the light, and the plurality of plasma characteristic information from the optical emission spectroscopy data and detecting the thin film characteristic information including at least one of deposition thickness information, refractive index information, and hydrogen concentration information of the thin film formed on the fabricating substrate using calculation equations.

According to an embodiment of the disclosure, the optical data analysis device may detect the plurality of plasma characteristic information including the wavelength distribution of the light, the intensity distribution of the light, temperature characteristic change information of an inert gas, and molecule temperature characteristic change information of the inert gas from the optical emission spectroscopy data, calculate a hydrogen concentration of the thin film formed on the fabricating substrate in real time by using the optical emission spectroscopy data, the plurality of plasma characteristic information, and a hydrogen concentration detection calculation equation, and calculate a thickness of the thin film formed on the fabricating substrate in real time by using deposition environment information including temperature information and pressure information in the chamber, the plurality of plasma characteristic information, and a thin film thickness detection calculation equation.

According to an embodiment of the disclosure, the hydrogen concentration detection calculation equation may be HIC(hp)=[Int.A]ⁿ×[Int.B]^{m}×EXP^{(-ke/Te)}×EXP^{(-kg/Tg)}, HIC(hp) may be the hydrogen concentration, and Int.A may be an intensity or a detection wavelength of the light detected from the optical emission spectroscopy data of a first deposition source gas, Int.B may be an intensity or a detection wavelength of light detected from the optical emission spectroscopy data of a second deposition source gas, n may be a first concentration weight, and m may be a second concentration weight, Tg may be a temperature of the inert gas that is a first plasma characteristic factor, and Te may be a molecule temperature of the inert gas that is a second plasma characteristic factor, and ke may be a first vibration energy weight, and kg may be a second vibration energy weight.

According to an embodiment of the disclosure, the optical data analysis device may detect a deposition rate of the thin film using the temperature information and the pressure information in the chamber, intensity characteristics of the light detected from the optical emission spectroscopy data of a first deposition source gas, and a deposition rate detection calculation equation, the deposition rate detection calculation equation may be THL(T_S)=k(p, T)[Int.A], and THL(T_S) may be deposition rate information of the thin film, p may be a pressure in the chamber, T may be a temperature in the chamber, and Int.A may be the intensity characteristics of the light detected from the optical emission spectroscopy data of the first deposition source gas.

According to an embodiment of the disclosure, the optical data analysis device may derive the thin film thickness detection calculation equation by integrating the deposition rate detection calculation equation with respect to time, the thin film thickness detection calculation equation may be THL(th)=k(p, T, . . . )∫₀^{t}Int.A dt

THL(th) may be the thickness of the thin film, and k may be a constant including 1.

According to an embodiment of the disclosure, an apparatus for fabricating a display panel may include a thin film deposition device forming a thin film on a fabricating substrate, and a thin film characteristic detection device generating optical emission spectroscopy data by analyzing a spectrum of light emitted during a thin film deposition process of the thin film deposition device and detecting thin film characteristic information of the thin film formed on the fabricating substrate by analyzing the optical emission spectroscopy data in real time. The thin film characteristic detection device may include a photosensitivity detection module generating the optical emission spectroscopy data based on a wavelength distribution and an intensity distribution of the light for each wavelength band emitted from an inside of a chamber of the thin film deposition device and an optical data analysis device analyzing the wavelength distribution of the light, the intensity distribution of the light, and a plurality of plasma characteristic information from the optical emission spectroscopy data and detecting the thin film characteristic information including at least one of deposition thickness information, refractive index information, and hydrogen concentration information of the thin film formed on the fabricating substrate using calculation equations.

According to an embodiment of the disclosure, the optical data analysis device may include a plasma characteristic extraction module detecting the plurality of plasma characteristic information including the wavelength distribution of the light, the intensity distribution of the light, temperature characteristic change information of an inert gas, and molecule temperature characteristic change information of the inert gas from the optical emission spectroscopy data, a hydrogen concentration detection module calculating a hydrogen concentration of the thin film formed on the fabricating substrate in real time by using the optical emission spectroscopy data, the plurality of plasma characteristic information, and a hydrogen concentration detection calculation equation, and a deposition film characteristic detection module calculating a thickness of the thin film formed on the fabricating substrate in real time by using deposition environment information including temperature information and pressure information in the chamber, the plurality of plasma characteristic information, and a thin film thickness detection calculation equation.

According to an embodiment of the disclosure, the optical data analysis device may detect the plurality of plasma characteristic information including the wavelength distribution of the light, the intensity distribution of the light, temperature characteristic change information of an inert gas, and molecule temperature characteristic change information of the inert gas from the optical emission spectroscopy data, calculate a hydrogen concentration of the thin film formed on the fabricating substrate in real time by using the optical emission spectroscopy data, the plurality of plasma characteristic information, and a hydrogen concentration detection calculation equation, and calculate a thickness of the thin film formed on the fabricating substrate in real time by using deposition environment information including temperature information and pressure information in the chamber, the plurality of plasma characteristic information, and a thin film thickness detection calculation equation.

According to an embodiment of the disclosure, the hydrogen concentration detection calculation equation may be HIC(hp)=[Int.A]ⁿ×[Int.B]^{m}×EXP^{(-ke/Te)}×EXP^{(-kg/Tg)}, HIC(hp) may be the hydrogen concentration, and Int.A may be an intensity or a detection wavelength of the light detected from the optical emission spectroscopy data of a first deposition source gas, Int.B may be an intensity or a detection wavelength of light detected from the optical emission spectroscopy data of a second deposition source gas, n may be a first concentration weight, and m may be a second concentration weight, Tg may be a temperature of the inert gas that is a first plasma characteristic factor, and Te may be a molecule temperature of the inert gas that is a second plasma characteristic factor, and ke may be a first vibration energy weight, and kg may be a second vibration energy weight.

According to an embodiment of the disclosure, the optical data analysis device may detect a deposition rate of the thin film using the temperature information and the pressure information in the chamber, intensity characteristics of the light detected from the optical emission spectroscopy data of a first deposition source gas, and a deposition rate detection calculation equation, the deposition rate detection calculation equation may be THL(T_S)=k(p, T, . . . )[Int.A], and THL(T_S) may be deposition rate information of the thin film, p may be a pressure in the chamber, T may be a temperature in the chamber, and Int.A may be the intensity characteristics of the light detected from the optical emission spectroscopy data of the first deposition source gas.

According to an embodiment of the disclosure, the optical data analysis device may derive the thin film thickness detection calculation equation by integrating the deposition rate detection calculation equation with respect to time, the thin film thickness detection calculation equation may be THL(th)−k(p, T, . . . )∫₀^{t}Int.A dt

THL(th) may be the thickness of the thin film, and k may be a constant including 1.

With an apparatus for fabricating a display panel according to an embodiment, it is possible to form thin films having accurately targeted film thicknesses by detecting a deposition thickness and a refractive index of a thin film deposited on a fabricating substrate, hydrogen concentration characteristics in the thin film, and the like, in real time. Accordingly, it is possible to simplify a fabricating process of the display panels and reduce a fabricating cost of the display panels.

It is possible to increase accuracy and reliability of the fabricating process of the display panel by confirming plasma characteristics and thin film deposition characteristics in real time and correcting a thin film deposition process in real time based on confirmation result data.

The effects of the disclosure are not limited to the aforementioned effects, and various other effects are included in the specification.

### BRIEF DESCRIPTION OF THE DRAWINGS

The above and other aspects and features of the disclosure will become more apparent by describing in detail embodiments thereof with reference to the attached drawings, in which:

FIG. 1 is a schematic cross-sectional view illustrating components of an apparatus for fabricating a display panel according to an embodiment of the disclosure;

FIG. 2 is a schematic block diagram illustrating components of an optical data analysis device illustrated in FIG. 1;

FIG. 3 is a flowchart illustrating a thin film characteristic detection process of a thin film characteristic detection device;

FIG. 4 is a thin film measurement result analysis graph for describing a thin film characteristic analysis and detection method; and

FIG. 5 is a thin film measurement result analysis graph for describing a thin film characteristic analysis and detection method.

### DETAILED DESCRIPTION OF THE EMBODIMENTS

The disclosure will now be described more fully hereinafter with reference to the accompanying drawings, in which embodiments of the disclosure are shown. This disclosure may, however, be embodied in different forms and should not be construed as limited to the embodiments set forth herein. Rather, these embodiments are provided so that this disclosure will be thorough and complete, and will fully convey the scope of the disclosure to those skilled in the art.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. The same reference numbers indicate the same components throughout the specification.

It will be understood that, although the terms “first,” “second,” etc. may be used herein to describe various elements, these elements should not be limited by these terms. These terms are only used to distinguish one element from another element. For instance, a first element discussed below could be termed a second element without departing from the teachings of the disclosure. Similarly, the second element could also be termed the first element.

Each of the features of the various embodiments of the disclosure may be combined or combined with each other, in part or in whole, and technically various interlocking and driving are possible. Each embodiment may be implemented independently of each other or may be implemented together in an association.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the exemplary term “below” can encompass both an orientation of above and below. Furthermore, the apparatus may be otherwise oriented (e.g., rotated 90 degrees or at other orientations), and, as such, the spatially relative descriptors used herein interpreted accordingly.

Unless otherwise defined or implied herein, all terms (including technical and scientific terms) used have the same meaning as commonly understood by those skilled in the art to which this disclosure pertains. It will be further understood that terms, such as those defined in commonly used dictionaries, should be interpreted as having a meaning that is consistent with their meaning in the context of the relevant art and should not be interpreted in an ideal or excessively formal sense unless clearly defined in the specification.

Hereinafter, specific embodiments will be described with reference to the accompanying drawings.

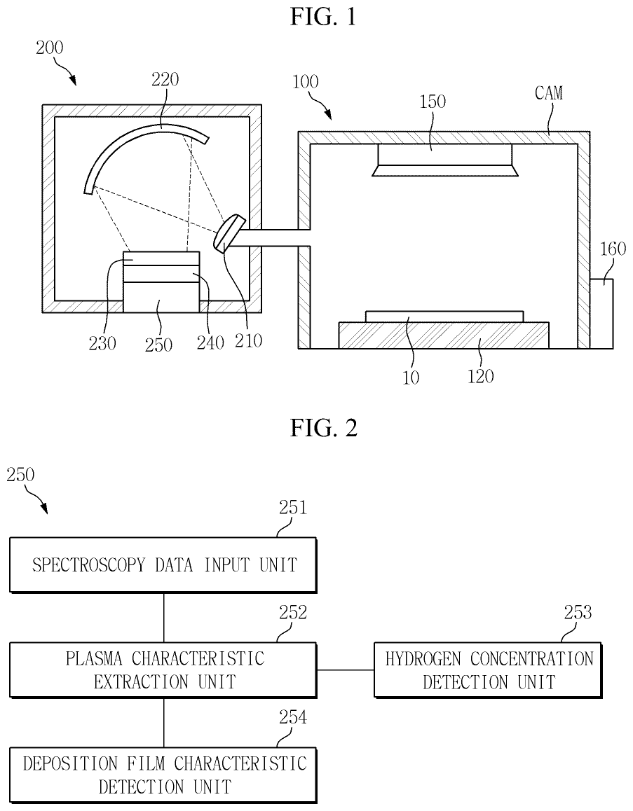

FIG. 1 is a schematic cross-sectional view illustrating components of an apparatus for fabricating a display panel according to an embodiment of the disclosure.

Referring to FIG. 1, the apparatus for fabricating a display panel may include a thin film deposition device 100 forming a thin film on a transparent insulating substrate (hereinafter also referred to as a fabricating substrate 10) and a thin film characteristic detection device 200 detecting characteristic information of the thin film formed on the fabricating substrate 10 in real time.

The thin film deposition device 100 may form a thin film on the fabricating substrate 10 made of silicon, glass, or the like, using photoresists or organic or inorganic insulating materials.

The thin film deposition device 100 may include a chemical vapor deposition device or the like that performs a plasma-enhanced chemical vapor deposition (PECVD) process. For example, the thin film deposition device 100 may include a chamber CAM, a loading plate 120, a gas supplier 150, and a plasma process controller 160.

The chamber CAM of the thin film deposition device 100 may form a space for performing a chemical vapor deposition process. The chamber CAM may be a PECVD process chamber, and may provide a process space having functions such as vacuum, heating, soundproofing, cooling, non-vibration, and/or waterproofing. To this end, a vacuum device, an air suction device, a purification device, a heating device, a cooling device, and/or the like, may be installed inside and/or outside the chamber CAM.

The loading plate 120 may be disposed in an internal space of the chamber CAM and fix the fabricating substrate 10 seated on a front surface of the loading plate 120. The loading plate 120 may include a heat generating member, an adsorption member, an alignment mark, and the like.

The gas supplier 150 may supply a deposition source gas and a carrier gas to the internal space of the chamber CAM. The deposition source gas may be a gas containing photoresists or organic or inorganic insulating materials, and the organic or inorganic insulating materials or the like may be formed as a thin film on the fabricating substrate 10 through a plasma treatment process. For example, the deposition source gas may be formed as a silicon oxide layer on the fabricating substrate 10 through a plasma process by including a silane-based gas and oxygen (O₂). The carrier gas may be an inert gas, and may include a helium (He) gas, an argon (Ar) gas, or the like.

The plasma process controller 160 may supply high-frequency power to the chamber CAM in order to activate the deposition source gas and the carrier gas into a plasma state, and control a plasma deposition process by setting a magnitude of the high-frequency power, a process time, and the like. For example, the plasma process controller 160 may control the plasma deposition process in real time by setting and changing thin film deposition process condition such as a pressure in the chamber CAM, a temperature in the chamber CAM, a flow rate of each gas, and high-frequency power condition information. The plasma process controller 160 may share preset or real-time variable thin film deposition process condition information and control information with the thin film characteristic detection device 200.

The thin film characteristic detection device 200 may generate optical emission spectroscopy data by analyzing a spectrum of light emitted during a thin film deposition process of the thin film deposition device 100. The thin film characteristic detection device 200 may detect thin film characteristic information of the thin film formed on the fabricating substrate 10 in real time by analyzing the optical emission spectroscopy data. For example, the thin film characteristic detection device 200 may detect a deposition thickness, a refractive index and hydrogen concentration information of the thin film formed on the fabricating substrate 10 in real time by analyzing a wavelength distribution of the light, an intensity distribution of the light, and multiple plasma characteristic information from the optical emission spectroscopy data.

A deposition process using plasma may be a process in which a radical generation reaction and a thin film deposition reaction occur repeatedly. In the plasma deposition process, the radical generation reaction may be performed relatively slowly compared to the thin film deposition reaction, and thus, a radical generation reaction rate may become a dominant factor in a deposition rate of the thin film. However, a factor having a dominant influence on the deposition rate of the thin film may be changed depending on a type of thin film that is deposited, a deposition method of the thin film, and the like. Accordingly, the deposition rate of the thin film may be calculated using the radical generation reaction rate having the greatest influence on the deposition rate of the thin film.

In the deposition process, excitation and relaxation phenomena in an activated state may continuously occur due to plasma discharge. The plasma may emit light. A wavelength and an intensity of the emitted light may be related to a progress state and an environment of the deposition process. Accordingly, the thin film characteristic detection device 200 may measure the optical emission spectroscopy data including an intensity distribution of the light for each wavelength in order to detect a progress of the plasma deposition process.

The thin film characteristic detection device 200 may detect an intensity of light for each wavelength of split light by analyzing the spectrum of the light emitted during the thin film deposition process of the thin film deposition device 100. The thin film characteristic detection device 200 may generate optical emission spectroscopy data of the plasma according to an intensity distribution of light for each wavelength according to the inert gas. The optical emission spectroscopy data calculated in real time may be basic data for measuring a thickness, a refractive index, and a hydrogen concentration of the deposited thin film in real time.

For example, the thin film characteristic detection device 200 may confirm plasma characteristic change information according to the plasma deposition process, such as temperature characteristic change information of the inert gas and molecule temperature characteristic change information of the inert gas. The thin film characteristic detection device 200 may calculate the hydrogen concentration of the deposited thin film in real time by using the optical emission spectroscopy data of the plasma, the plasma characteristic change information, and a hydrogen concentration detection calculation equation.

In the deposition process using the plasma, the optical emission spectroscopy data at a wavelength corresponding to the inert gas may be proportional to the radical generation reaction rate. The optical emission spectroscopy data at the wavelength corresponding to the inert gas may be proportional to an excitation rate of the plasma and a concentration of the plasma. Since the radical generation reaction rate has a dominant influence on the deposition rate of the thin film, the optical emission spectroscopy data of the inert gas may be used as basic data for measuring the deposition rate and the deposition thickness of the thin film. The deposition rate of the thin film according to a plasma deposition process condition may be dominantly influenced by the deposition source gas or be dominantly influenced by each of the deposition source gas and the inert gas. Therefore, optical emission spectroscopy data for the deposition source gas may be detected and used to calculate the deposition rate of the thin film and the deposition thickness of the thin film. For example, the thin film characteristic detection device 200 may calculate a thickness of the deposited thin film in real time by using the optical emission spectroscopy data of the plasma, the temperature characteristic change information of the inert gas, deposition environment information such as the temperature and the pressure in the chamber CAM, and a thin film thickness detection calculation equation. The thin film characteristic detection device 200 may confirm the refractive index of the thin film by material information and the thickness information of the thin film, and generate data including thickness distribution information, thickness uniformity information, and the like, of the thin film.

Referring to FIG. 1, the thin film characteristic detection device 200 may be a component for generating the optical emission spectroscopy data by analyzing the spectrum of the light emitted during the plasma deposition process, and include a full-range spectroscopy module 210, a diffraction module 220, an incident light filter module 230, a photosensitivity detection module 240, and an optical data analysis device 250.

The full-range spectroscopy module 210 may capture plasma light particles emitted from the inside of the chamber CAM of the thin film deposition device 100 and split and refract plasma light according to a spectrum wavelength band. For example, the full-range spectral module 210 may include a lens module in which at least one concave lens or convex lens is combined, at least one prism lens which refracts light for each wavelength band at a preset angle and emits the light, and the like.

The diffraction module 220 may reflect the light of the spectral wavelength band refracted from the full-range spectroscopy module 210 at a reflection angle according to an incident angle. The diffraction module 220 may include a flat plate disposed to form a reflection angle with a gradient, an antenna-type plate having a concave reflective surface, a concave hemispherical disk or plate, or the like.

The incident light filter module 230 may band-pass-filter the light of the spectral wavelength band reflected from the diffraction module 220 in a wavelength band. The incident light filter module 230 may include multiple band pass filters filtering light of a wavelength band.

The photosensitivity detection module 240 may generate optical emission spectroscopy data based on a wavelength distribution and an intensity distribution of the light for each wavelength band of the plasma light incident through the incident light filter module 230. The photosensitivity detection module 240 may include at least one optical emission spectroscopy. As described above, the photosensitivity detection module 240 may generate the optical emission spectroscopy data by splitting light emitted from the inert gas and measuring intensities of the light for each wavelength of the split light and aligning the intensities of the light with each other.

The optical data analysis device 250 may analyze the wavelength distribution of the light, the intensity distribution of the light, and the plasma characteristic information from the optical emission spectroscopy data. The optical data analysis device 250 may detect at least one of thin film characteristic information of deposition thickness information, refractive index information, and hydrogen concentration information of the thin film formed on the fabricating substrate 10 in real time using optical characteristic information and plasma characteristic information analyzed from the optical emission spectroscopy data and a calculation equation.

FIG. 2 is a schematic block diagram illustrating components of an optical data analysis device illustrated in FIG. 1.

Referring to FIG. 2, the optical data analysis device 250 may include a spectroscopy data input unit (or module) 251, a plasma characteristic extraction unit (or module) 252, a hydrogen concentration detection unit (or module) 253, and a deposition film characteristic detection unit (or module) 254.

The spectroscopy data input unit 251 may receive the optical emission spectroscopy data in real time from the photosensitivity detection module 240 and store the optical emission spectroscopy data in a memory. The spectroscopy data input unit 251 may share the stored optical emission spectroscopy data with the plasma characteristic extraction unit 252 and the like.

The plasma characteristic extraction unit 252 may analyze wavelength distribution information of the light and intensity distribution information of the light from the optical emission spectroscopy data and convert the analyzed information into data. The plasma characteristic extraction unit 252 may confirm intensity distribution information of light by aligning intensities of light generated from main radicals contributing to the deposition process with each other based on a time and a space. The plasma characteristic extraction unit 252 may confirm a wavelength distribution of the light by aligning an intensity distribution and a wavelength change of the light based on intensity versus time. The plasma characteristic extraction unit 252 may detect multiple plasma characteristic information such as temperature characteristic change information of the inert gas and molecule temperature characteristic change information of the inert gas from the optical emission spectroscopy data.

The plasma characteristic extraction unit 252 may read and pre-store reference temperature information corresponding to intensity information of the light versus a magnitude of vibration energy from a memory or the like. The plasma characteristic extraction unit 252 may confirm a change in vibration energy detected in the carrier gas, and confirm a change in intensity distribution of the light according to a change in magnitude of the vibration energy. The plasma characteristic extraction unit 252 may extract temperature information of the inert gas in real time using the reference temperature information corresponding to the magnitude of the vibration energy and the intensity information of the light detected in the carrier gas in real time.

The plasma characteristic extraction unit 252 may read and pre-store rovibrational energy distribution information for each wavelength band and molecule temperature information for each wavelength band of the carrier gas used in the deposition process from the memory or the like depending on a type of the carrier gas. Accordingly, the plasma characteristic extraction unit 252 may confirm a change in vibration energy detected in the carrier gas in real time and detect a wavelength band according to a change in magnitude of the vibration energy. The plasma characteristic extraction unit 252 may confirm and extract a molecule temperature according to the corresponding wavelength range.

The hydrogen concentration detection unit 253 may calculate a hydrogen concentration of the deposited thin film in real time by using the optical emission spectroscopy data of the plasma and the plasma characteristic information using a hydrogen concentration detection calculation equation.

The hydrogen concentration detection calculation equation may be preset and stored as follows.

$$[ Hydrogen concentration detection calculation equation ] \\ HIC ( h p ) = {[ Int . A ]}^{n} \times {[ Int . B ]}^{m} \times {EXP}^{( - ke / Te )} \times {EXP}^{( - kg / Tg )}$$

HIC(hp) may be a hydrogen concentration, Int.A may be an optical characteristic (e.g., an intensity or a detection wavelength of light) detected from optical emission spectroscopy data of a first deposition source gas, and Int.B may be an optical characteristic (e.g., an intensity or a detection wavelength of light) detected from optical emission spectroscopy data of a second deposition source gas.

n may be a first concentration weight, and m may be a second concentration weight. Tg may be a first plasma characteristic factor (e.g., a temperature of the inert gas), and Te may be a second plasma characteristic factor (e.g., a molecule temperature of the inert gas). ke may be a first energy weight (e.g., a vibration energy weight), and kg may be a second energy weight (e.g., a vibration energy weight).

The deposition film characteristic detection unit 254 may calculate thickness of the deposited thin film in real time by using deposition environment information including temperature information and pressure information in the chamber CAM and multiple plasma characteristic information using a thin film thickness detection calculation equation. For example, the deposition film characteristic detection unit 254 may first detect a deposition rate of the thin film using the temperature information and the pressure information in the chamber CAM, optical characteristics (e.g., intensity characteristics of the light) detected from the optical emission spectroscopy data of the first deposition source gas, and a deposition rate detection calculation equation.

The deposition rate detection calculation equation of the thin film may be preset and stored as follows.

$$[ Deposition rate detection calculation equation ] \\ THL ( T\_S ) = k ( p , T , … ) [ Int . A ]$$

THL(T_S) may be deposition rate information of the thin film, p may be a pressure in the chamber CAM, T may be a temperature in the chamber CAM, and Int.A may be optical characteristic information (e.g., intensity information of light) detected from the optical emission spectroscopy data of the first deposition source gas.

By integrating the deposition rate detection calculation equation of the thin film with respect to time, a thin film thickness detection calculation equation may be derived.

The thin film thickness detection calculation equation may be preset and stored as follows.

$$THL ( th ) = k ( p , T , … ) {\int }_{0}^{t} Int . A dt$$

THL(th) may be a calculated thickness value of the thin film. k may be a constant including 1. The deposition film characteristic detection unit 254 may confirm the refractive index of the thin film through material information and the thickness information of the thin film, and generate data including thickness distribution information, thickness uniformity information, and the like, of the thin film.

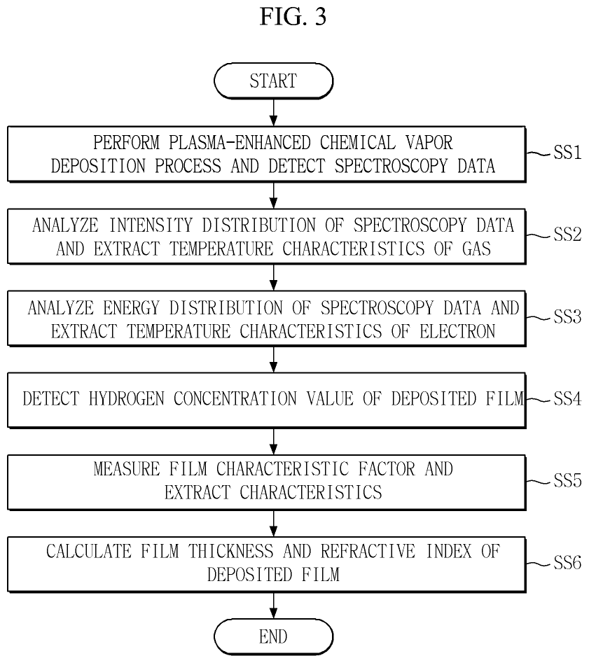

FIG. 3 is a flowchart illustrating a thin film characteristic detection process of a thin film characteristic detection device.

Referring to FIG. 3, the plasma process controller 160 may apply high-frequency power to the chamber CAM in order to activate the deposition source gas and the carrier gas into a plasma state, and control a plasma deposition process by setting a magnitude of the high-frequency power, a process time, and the like. For example, the plasma process controller 160 may control the plasma deposition process in real time by setting and changing thin film deposition process condition such as a pressure in the chamber CAM, a temperature in the chamber CAM, a flow rate of each gas, and high-frequency power condition. The thin film characteristic detection device 200 may generate optical emission spectroscopy data by analyzing a spectrum of light emitted during a thin film deposition process of the thin film deposition device 100 (SS1).

The plasma characteristic extraction unit 252 of the thin film characteristic detection device 200 may detect intensity distribution information of light by aligning intensities of light generated from main radicals contributing to a deposition process with each other based on a time and a space, and detect a wavelength distribution of the light by aligning an intensity distribution and a wavelength change of the light based on intensity versus time (SS2).

The plasma characteristic extraction unit 252 may extract temperature information of the inert gas in real time using reference temperature information corresponding to a magnitude of vibration energy and intensity information of light detected in the carrier gas in real time. Subsequently, the plasma characteristic extraction unit 252 may confirm a change in vibration energy detected in the carrier gas in real time and detect a wavelength band according to a change in magnitude of the vibration energy. The plasma characteristic extraction unit 252 may confirm and extract a molecule temperature according to the corresponding wavelength range (SS3).

The plasma characteristic extraction unit 252 may measure a hydrogen concentration of the thin film in real time while plasma deposition is being performed, using a hydrogen concentration detection calculation equation. For example, the plasma characteristic extraction unit 252 may measure a hydrogen concentration of the thin film in real time by substituting temperature characteristics of the inert gas and molecule temperature characteristics of the inert gas into the hydrogen concentration detection calculation equation (SS4).

The plasma characteristic extraction unit 252 may first detect deposition rate information of the thin film using a deposition rate detection calculation equation of the thin film in order to detect thickness information of the thin film, which is one of thin film characteristic factors, in real time. By integrating the deposition rate detection calculation equation of the thin film with respect to time, a thin film thickness detection calculation equation may be derived. The thickness information of the thin film may be detected in real time as represented in Table 1 using the thin film thickness detection calculation equation. (SS5)

TABLE 1

| | | Integral | Measured | Actual | |
|---|---|---|---|---|---|
| | OES | OES Peak | Thickness | Deposition | |
| Sample | Intensity | ∫₀^{t} | (Å) k(p, T, . . . ) | Thickness | Error |
| Substrate | (I_{OES}) | I_{OES} dt | ∫₀^{t} I_{OES} dt | (Å) | Rate |
| 6 | 8136 | 315022 | 2032 | 2071 | −1.87 |
| 7 | 8105 | 318796 | 2049 | 2022 | 1.35 |
| 8 | 7552 | 290477 | 1921 | 1909 | 0.63 |
| 9 | 3870 | 166235 | 1359 | 1370 | −0.81 |
| 10 | 4890 | 232139 | 1657 | 1647 | 0.65 |

OES intensity may be an intensity of light detected from optical emission spectroscopy data of a first deposition source gas.

Thereafter, the deposition film characteristic detection unit 254 may confirm a refractive index of the thin film by comparing material information and thickness information of the thin film with refractive index reference information versus a thickness of a material. The deposition film characteristic detection unit 254 may calculate thickness distribution information of the thin film, thickness uniformity information according to a refractive index change, and the like, by converting a refractive index change or distribution for each position of the thin film into data in a plane distribution or a graph. Accordingly, characteristic data information including the thickness distribution information of the thin film, the thickness uniformity information according to the refractive index change, and the like, may be generated (SS6).

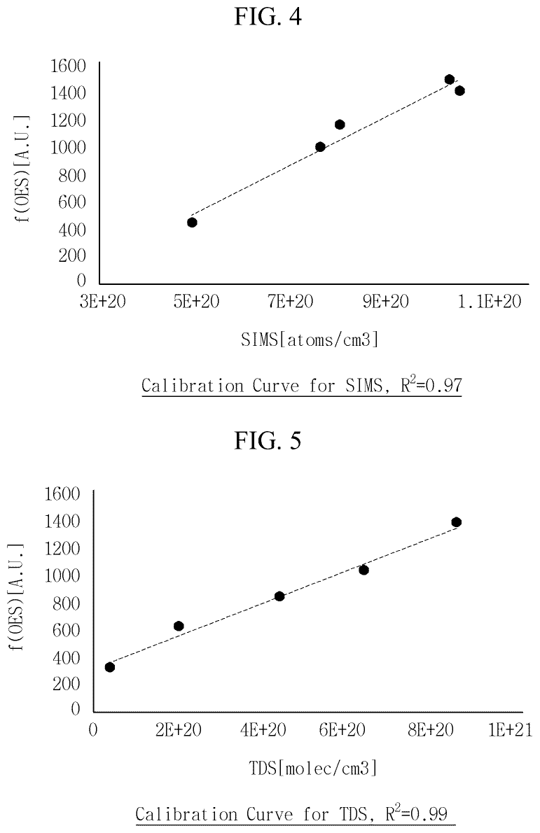

FIG. 4 is a thin film measurement result analysis graph for describing a thin film characteristic analysis and detection method.

Referring to FIG. 4, a silicon oxide film may be deposited in the chamber CAM using a silane-based gas and oxygen as first and second deposition source gases and using helium as an inert gas. It is possible to confirm an intensity distribution value (OSE) of light for each sampled detection position (SIMS) or a change in vibration energy for each sampled detection position (SIMS) versus an intensity value of the light or confirm an ionized molecule temperature. A calibration value (R²) for matching the ionized molecule temperature to the intensity distribution value (OSE) of the light for each sampled detection position (SIMS) may be preset according to an experimental value. As illustrated in FIG. 4, the hydrogen concentration detection unit 253 may detect a molecule temperature of the inert gas and apply the molecule temperature as a second plasma characteristic factor to a hydrogen concentration detection calculation equation.

FIG. 5 is a thin film measurement result analysis graph for describing a thin film characteristic analysis and detection method.

Referring to FIG. 5, a silicon oxide film may be deposited in the chamber CAM using a silane-based gas and oxygen as first and second deposition source gases and using helium as an inert gas. It is possible to confirm an intensity distribution value (OSE) of light for each light detection position for each sampled thin film material (TDS) or a change in deposition temperature for each sampled thin film material (TDS) versus an intensity value of the light or confirm an ionized molecule temperature. A calibration value (R²) for matching the deposition temperature for each sampled thin film material (TDS) or the ionized molecule temperature to the intensity distribution value (OSE) of the light for each sampled thin film material (TDS) may be preset according to an experimental value. As illustrated in FIG. 5, the hydrogen concentration detection unit 253 may detect a real-time temperature or a molecule temperature of the inert gas and apply the real-time temperature or the molecule temperature of the inert gas as a first or second plasma characteristic factor to a hydrogen concentration detection calculation equation.

With the apparatus for fabricating a display panel according to an embodiment as described above, it is possible to form thin films having accurately targeted film thicknesses by detecting the deposition thickness and the refractive index of the thin film deposited on the fabricating substrate 10, the hydrogen concentration in the thin film, and the like, in real time. Accordingly, it is possible to simplify a fabricating process of the display panels and reduce a fabricating cost of the display panels.

It is also possible to increase accuracy and reliability of the fabricating process of the display panel by confirming plasma characteristics and thin film deposition characteristics in real time and correcting a thin film deposition process in real time based on confirmation result data.

The above description is an example of technical features of the disclosure, and those skilled in the art to which the disclosure pertains will be able to make various modifications and variations. Therefore, the embodiments of the disclosure described above may be implemented separately or in combination with each other.

Therefore, the embodiments disclosed in the disclosure are not intended to limit the technical spirit of the disclosure, but to describe the technical spirit of the disclosure, and the scope of the technical spirit of the disclosure is not limited by these embodiments. The protection scope of the disclosure should be interpreted by the following claims, and it should be interpreted that all technical spirits within the equivalent scope are included in the scope of the disclosure.

What is claimed is:

1. An apparatus for fabricating a display panel, comprising:
a thin film deposition device forming a thin film on a fabricating substrate; and
a thin film characteristic detection device generating optical emission spectroscopy data by analyzing a spectrum of light emitted during a thin film deposition process of the thin film deposition device and detecting thin film characteristic information of the thin film formed on the fabricating substrate by analyzing the optical emission spectroscopy data in real time,
wherein the thin film characteristic detection device detects the thin film characteristic information including a hydrogen concentration of the thin film formed on the fabricating substrate by analyzing a wavelength distribution of the light, an intensity distribution of the light, and a plurality of plasma characteristic information from the optical emission spectroscopy data.

2. The apparatus for fabricating a display panel of claim 1, wherein the thin film deposition device includes:
a chamber forming a space for performing a chemical vapor deposition process;
a loading plate disposed in an internal space of the chamber and seating and fixing the fabricating substrate;
a gas supplier supplying a deposition source gas and a carrier gas to the internal space of the chamber; and
a plasma process controller applying high-frequency power to an inside of the chamber and controlling a plasma deposition process.

3. The apparatus for fabricating a display panel of claim 1, wherein the thin film characteristic detection device includes:
a full-range spectroscopy module splitting and refracting the light emitted from an inside of a chamber of the thin film deposition device according to a spectrum wavelength band;
a diffraction module reflecting the light refracted from the full-range spectroscopy module;
an incident light filter module band-pass-filtering the light reflected from the diffraction module in a wavelength band;
a photosensitivity detection module generating the optical emission spectroscopy data based on a wavelength distribution and an intensity distribution of the light for each wavelength band of the light filtered through the incident light filter module; and
an optical data analysis device analyzing the wavelength distribution of the light, the intensity distribution of the light, and the plurality of plasma characteristic information from the optical emission spectroscopy data and detecting the thin film characteristic information including at least one of deposition thickness information, refractive index information, and hydrogen concentration information of the thin film formed on the fabricating substrate using calculation equations.

4. The apparatus for fabricating a display panel of claim 3, wherein the optical data analysis device includes:
a plasma characteristic extraction module detecting the plurality of plasma characteristic information including the wavelength distribution of the light, the intensity distribution of the light, temperature characteristic change information of an inert gas, and molecule temperature characteristic change information of the inert gas from the optical emission spectroscopy data;
a hydrogen concentration detection module calculating a hydrogen concentration of the thin film formed on the fabricating substrate in real time by using the optical emission spectroscopy data, the plurality of plasma characteristic information, and a hydrogen concentration detection calculation equation; and
a deposition film characteristic detection module calculating a thickness of the thin film formed on the fabricating substrate in real time by using deposition environment information including temperature information and pressure information in the chamber, the plurality of plasma characteristic information, and a thin film thickness detection calculation equation.

5. The apparatus for fabricating a display panel of claim 1, wherein the thin film characteristic detection device includes:
a photosensitivity detection module generating the optical emission spectroscopy data based on a wavelength distribution and an intensity distribution of the light for each wavelength band emitted from an inside of a chamber of the thin film deposition device; and
an optical data analysis device analyzing the wavelength distribution of the light, the intensity distribution of the light, and the plurality of plasma characteristic information from the optical emission spectroscopy data and detecting the thin film characteristic information including at least one of deposition thickness information, refractive index information, and hydrogen concentration information of the thin film formed on the fabricating substrate using calculation equations.

6. The apparatus for fabricating a display panel of claim 5, wherein the optical data analysis device:
detects the plurality of plasma characteristic information including the wavelength distribution of the light, the intensity distribution of the light, temperature characteristic change information of an inert gas, and molecule temperature characteristic change information of the inert gas from the optical emission spectroscopy data;
calculates a hydrogen concentration of the thin film formed on the fabricating substrate in real time by using the optical emission spectroscopy data, the plurality of plasma characteristic information, and a hydrogen concentration detection calculation equation; and
calculates a thickness of the thin film formed on the fabricating substrate in real time by using deposition environment information including temperature information and pressure information in the chamber, the plurality of plasma characteristic information, and a thin film thickness detection calculation equation.

7. The apparatus for fabricating a display panel of claim 6, wherein
the hydrogen concentration detection calculation equation is HIC(hp)=[Int.A]ⁿ×[Int.B]^{m}×EXP^{(-ke/Te)}×EXP^{(-kg/Tg)},
HIC(hp) is the hydrogen concentration, and Int.A is an intensity or a detection wavelength of the light detected from the optical emission spectroscopy data of a first deposition source gas,
Int.B is an intensity or a detection wavelength of light detected from the optical emission spectroscopy data of a second deposition source gas, n is a first concentration weight, and m is a second concentration weight,
Tg is a temperature of the inert gas that is a first plasma characteristic factor, and Te is a molecule temperature of the inert gas that is a second plasma characteristic factor, and
ke is a first vibration energy weight, and kg is a second vibration energy weight.

8. The apparatus for fabricating a display panel of claim 6, wherein
the optical data analysis device detects a deposition rate of the thin film using the temperature information and the pressure information in the chamber, intensity characteristics of the light detected from the optical emission spectroscopy data of a first deposition source gas, and a deposition rate detection calculation equation,
the deposition rate detection calculation equation is THL(T_S)=k(p, T)[Int.A], and
THL(T_S) is deposition rate information of the thin film, p is a pressure in the chamber, T is a temperature in the chamber, and Int.A is the intensity characteristics of the light detected from the optical emission spectroscopy data of the first deposition source gas.

9. The apparatus for fabricating a display panel of claim 8, wherein
the optical data analysis device derives the thin film thickness detection calculation equation by integrating the deposition rate detection calculation equation with respect to time, and
the thin film thickness detection calculation equation is THL(th)=k(p, T, . . . )∫₀^{t}Int.A dt
wherein THL(th) is the thickness of the thin film, and k is a constant including 1.

10. An apparatus for fabricating a display panel, comprising:
a thin film deposition device forming a thin film on a fabricating substrate; and
a thin film characteristic detection device generating optical emission spectroscopy data by analyzing a spectrum of light emitted during a thin film deposition process of the thin film deposition device and detecting thin film characteristic information of the thin film formed on the fabricating substrate by analyzing the optical emission spectroscopy data in real time, wherein the thin film characteristic detection device includes:
a photosensitivity detection module generating the optical emission spectroscopy data based on a wavelength distribution and an intensity distribution of the light for each wavelength band emitted from an inside of a chamber of the thin film deposition device; and
an optical data analysis device analyzing the wavelength distribution of the light, the intensity distribution of the light, and a plurality of plasma characteristic information from the optical emission spectroscopy data and detecting the thin film characteristic information including at least one of deposition thickness information, refractive index information, and hydrogen concentration information of the thin film formed on the fabricating substrate using calculation equations.

11. The apparatus for fabricating a display panel of claim 10, wherein the optical data analysis device includes:
a plasma characteristic extraction module detecting the plurality of plasma characteristic information including the wavelength distribution of the light, the intensity distribution of the light, temperature characteristic change information of an inert gas, and molecule temperature characteristic change information of the inert gas from the optical emission spectroscopy data;
a hydrogen concentration detection module calculating a hydrogen concentration of the thin film formed on the fabricating substrate in real time by using the optical emission spectroscopy data, the plurality of plasma characteristic information, and a hydrogen concentration detection calculation equation; and
a deposition film characteristic detection module calculating a thickness of the thin film formed on the fabricating substrate in real time by using deposition environment information including temperature information and pressure information in the chamber, the plurality of plasma characteristic information, and a thin film thickness detection calculation equation.

12. The apparatus for fabricating a display panel of claim 10, wherein the optical data analysis device:
detects the plurality of plasma characteristic information including the wavelength distribution of the light, the intensity distribution of the light, temperature characteristic change information of an inert gas, and molecule temperature characteristic change information of the inert gas from the optical emission spectroscopy data;
calculates a hydrogen concentration of the thin film formed on the fabricating substrate in real time by using the optical emission spectroscopy data, the plurality of plasma characteristic information, and a hydrogen concentration detection calculation equation; and
calculates a thickness of the thin film formed on the fabricating substrate in real time by using deposition environment information including temperature information and pressure information in the chamber, the plurality of plasma characteristic information, and a thin film thickness detection calculation equation.

13. The apparatus for fabricating a display panel of claim 12, wherein
the hydrogen concentration detection calculation equation is HIC(hp)=[Int.A]ⁿ×[Int.B]^{m}×EXP^{(-ke/Te)}×EXP^{(-kg/Tg)},
HIC(hp) is the hydrogen concentration, and Int.A is an intensity or a detection wavelength of the light detected from the optical emission spectroscopy data of a first deposition source gas,
Int.B is an intensity or a detection wavelength of light detected from the optical emission spectroscopy data of a second deposition source gas, n is a first concentration weight, and m is a second concentration weight,
Tg is a temperature of the inert gas that is a first plasma characteristic factor, and Te is a molecule temperature of the inert gas that is a second plasma characteristic factor, and
ke is a first vibration energy weight, and kg is a second vibration energy weight.

14. The apparatus for fabricating a display panel of claim 12, wherein
the optical data analysis device detects a deposition rate of the thin film using the temperature information and the pressure information in the chamber, intensity characteristics of the light detected from the optical emission spectroscopy data of a first deposition source gas, and a deposition rate detection calculation equation,
the deposition rate detection calculation equation is THL(T_S)=k(p, T)[Int.A], and
THL(T_S) is deposition rate information of the thin film, p is a pressure in the chamber, T is a temperature in the chamber, and Int.A is the intensity characteristics of the light detected from the optical emission spectroscopy data of the first deposition source gas.

15. The apparatus for fabricating a display panel of claim 14, wherein
the optical data analysis device derives the thin film thickness detection calculation equation by integrating the deposition rate detection calculation equation with respect to time, and
the thin film thickness detection calculation equation is THL(th)=k(p, T, . . . )∫₀^{t}Int.A dt
wherein THL(th) is the thickness of the thin film, and k is a constant including 1.
